(19) United States
(12) Patent Application Publication
(10) Pub. No.: US 2009/0010970 A1
(43) Pub. Date: Jan. 8, 2009

(54) DISSOLVABLE TOOTH WHITENING STRIP

(76) Inventor: Jose Luis Velada, Burgos (ES)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 11/910,298

(22) PCT Filed: Oct. 4, 2005
(86) PCT No.: PCT/US2005/035518

§ 371 Date: Apr. 4, 2008

(30) Foreign Application Priority Data
Mar. 31, 2005 (US) PCT US05 010941

(51) Int. Cl. A61K 8/24; A61K 8/02

(52) U.S. Cl. 424/401; 424/57

(57) ABSTRACT

The present invention provides a dissolvable strip for whitening teeth. The strip, which is preferably a single layer, has a whitening agent and a water-soluble or water dispersible polymer system. The dissolution of the whitening composition is controlled by interaction of the whitening composition with an oral environment containing saliva. The present invention further provides a process for preparing the whitening strip in the form of a dry film and a method of whitening teeth.

### BACKGROUND OF THE INVENTION

1. Field of the Invention

The present invention relates to a dissolvable whitening strip for whitening teeth. More particularly, the present invention relates to a dissolvable whitening strip having a whitening agent and a polymer system for a controlled, yet complete, dissolution of the whitening strip by interaction of the whitening strip with saliva in the oral cavity.

2. Description of Related Art

Several types of patches and other products for whitening teeth and drug delivery in an individual film-shaped dosage form are known in the art. Also known are powder compositions for whitening teeth having a fluoride-containing compound, an essential amino acid and a polyprotic acid.

Recently, commercially known whitening strips have been developed to whiten teeth. However, these commercial whitening strips are not pliable or flexible enough to contact all desired surfaces of the teeth in an oral cavity. These strips have the drawbacks of not contacting, and thus not efficiently whitening these areas, especially between teeth (interstices). If the strip is pressed to cover these interstices, these strips may lift from a portion of the teeth thereby not providing entirely uniform whitening.

Despite considerable interest in systems for whitening teeth, there is a need in the consumer products and dental care industries for products that can enhance the appearance of teeth by whitening all desired surfaces of the teeth. Preferably, they also deliver additional benefits to the consumer, yet provide convenience to the user. Such additional benefits preferably include increased opacity of the teeth, accelerated whitening of the teeth without the need for pre-mixing of reagents and activators, prevention of tooth sensitivity, and more precise control of diffusion rates of the one or more active ingredients and increasing the contact time with the teeth.

A whitening strip having a controlled-dissolution, in which the strip dissolves completely while on the teeth, and leaves virtually no undesired residue, is desired. The present invention provides such a whitening strip.

### SUMMARY OF THE INVENTION

It is an object of the present invention to provide a dissolvable strip for whitening teeth.

It is another object of the present invention to provide a dissolvable, single layer whitening strip having a whitening agent and a water-soluble or water dispersible polymer system.

It is still another object of the present invention to provide a whitening strip that dissolves in the mouth leaving virtually no undesired residue.

It is still yet another object of the present invention to provide a whitening strip that has controllable contact time.

It is yet another object of the present invention to provide a whitening strip that can be targeted to deliver the whitening agent to the desired area at a desired delivery rate.

It is a further object of the present invention to provide a dissolvable whitening strip that is initially a dry film, but hydrates thereby becoming gelatinous upon contact with saliva, and initiates its adhesiveness.

It is a still further object of the present invention to provide a dissolvable whitening strip that is a dry film for easy handling prior to use.

It is a yet further object of the present invention to provide such a dissolvable whitening strip that upon exposure to saliva commences hydration and, thus, becomes very pliable to conform to the configuration of the teeth thereby enhancing contact with the desired surface of the teeth.

It is still yet a further object of the present invention to provide such a dissolvable whitening strip in which its flexibility increases and its adhesiveness increases during continued exposure to saliva or hydration in the oral cavity.

It is a still further object of the present invention to provide such a dissolvable whitening strip that does not require a backing layer.

It is a still yet further object of the present invention to provide such a dissolvable whitening strip in which the dissolution rate is controlled by the composition of the strip matrix.

It is also an object of the present invention to provide a multi-layered whitening strip for whitening teeth in which the overall dissolution of the strip is controlled by interaction of each layer with saliva.

It is another object of the present invention to provide a multi-layered whitening strip for whitening teeth in which there are one or more zones in a layer of the strip having one or more different ingredients.

The present invention provides such a dissolvable, water-soluble strip for whitening teeth. The strip has a whitening agent and a water-soluble or water dispersible polymer system. The polymer system includes a poly(vinylpyrrolidone) or any derivative thereof, and at least one other polymer. The dissolution of said whitening strip is preferably controlled solely by interaction of the strip with an oral environment containing saliva.

The present invention also provides a method of whitening teeth, i.e., a method of improving the whiteness of the teeth. The method has the step of applying onto the teeth a whitening strip according to the present invention for a period of time sufficient to produce in the course of treatment a detectable, as known in the art, improvement in the whiteness of the teeth.

These and other objects and advantages of the present invention are achieved by the use of the whitening strip according to the present invention, which dissolves leaving virtually no undesired residue.

### DETAILED DESCRIPTION OF THE INVENTION

The present invention provides a dissolvable whitening strip or film. As used herein, whitening strip means the formed dry film. In the most preferred embodiment, the strip is a single layer, although in alternative embodiments, as noted below, the strip can be multi-layered.

The strip has a polymer system and a whitening agent or agents for improving the whitening of teeth. The whitening strip may have additional ingredients, some of which may be active, to provide additional benefits that include breath freshening, whitening maintenance, tooth mineralization, tooth sensitivity prevention or treatment, and gum health maintenance or treatment.

In any embodiment, the strip of the present invention is dissolvable in saliva that is formed in the oral cavity or mouth. In an alternative embodiment, after application of the strip onto teeth, a substantive coating, that is not considered undesired residue, can be left to extend the residence time of the whitening agent or any active ingredient.

The whitening strip is initially a film. However, once exposed to saliva in the oral cavity, the strip immediately, or virtually immediately, hydrates and becomes gelatinous. The hydration can result in a swelling or expansion of the gelatinous material. This gelatinous structure continues to hydrate, and thus becomes even more flexible during its time in the oral cavity until it dissolves with virtually no undesired residue. Accordingly, the strip becomes more and more flexible and pliable and, thus, continues to adapt to the contours or morphology of the teeth upon which it is applied. Also, the continuous hydration causes an increase in the adhesive properties of the strip, which enables the strip to adapt and adhere to the contours of the teeth, including filling into the cavities and the spaces or interstices between the teeth, even better to carry out its whitening action in all desired surfaces of the teeth. The ability of the present strip to cover, and thus in effect whiten, the interstices of teeth is a major advantage over prior art strips.

Significantly, once exposed to saliva in the oral cavity and as the duration of the exposure increases, the adhesiveness increases and, thus, the strength of the contact with the teeth increases. The increased adhesiveness improves the efficacy of the strip.

The solubility or dissolvability of the strip is controlled by the composition to enable dissolution of the strip or film over a desired pre-determined time frame. The time frame of the dissolution of the strip can be adjusted based on the end benefit desired. To adjust the time frame, the nature of the water-soluble or water dispersible polymer system, the degree of crosslinking, if any, and the thickness of the strip should be adjusted. Generally, the thickness of the strip is about 5 μm to about 2000 μm. Preferably, the thickness of the strip is about 50 μm to about 500 μm.

The present invention provides a whitening strip that can be targeted to deliver the whitening agent to the desired area at a desired delivery rate, for example, within about 1 minute to about 360 minutes, preferably from about 1 minute to about 30 minutes, and most preferably from about 5 minutes to about 20 minutes, to whiten the teeth.

In another embodiment, the whitening agent or another desired agent can be encapsulated in a water-soluble or water dispersible shell and incorporated within or on a surface of the whitening strip, or a layer of the whitening strip, to further control the delivery rate.

The whitening strip preferably does not have a backing or backing layer. A backing layer as defined in this application means the outermost layer opposite the layer contacting the tooth surface that is dissolvable and is a functional part of a multi-layer whitening strip. Preferably, the backing layer aids in comfort, such as, for example, it prevents unwanted adhesion to surfaces, other than the teeth, while in the oral cavity. If a backing layer is used in the whitening strip of the present invention, the backing layer must be completely dissolvable or water dispersible.

The whitening strip of the present invention has a polymer system that includes a first polymer, namely water-soluble or water dispersible poly-(vinylpyrrolidone)(PVP) or any of its derivatives, and at least one second polymer. The combination of the first and second polymers define the water-soluble and/or water swellable and/or water dispersible polymer system. The polymer system has adhesive properties, such that when it is brought to the teeth, the strip will adhere to the teeth.

The second polymer that can be used with the first polymer to produce the water-soluble or water dispersible polymer system according to the present invention, includes one or more of: an alkyl vinyl ether/maleic anhydride copolymer, alkyl vinyl ether/maleic acid copolymer, alkali metal or an amine salt of alkyl vinyl ether/maleic acid copolymer, partially or fully crosslinked alkyl vinyl ether/maleic anhydride copolymer, vinyl acetate copolymer, polyacrylates, polyurethane interpolymers, chitosan, poly(acrylic acid), poly(vinyl alcohol), poly(vinyl alcohol-g-ethylene glycol) copolymer, cellulose derivatives, hydroxy-propyl-methyl cellulose, hydroxyl-ethyl cellulose, hydroxy-propyl cellulose, poly(ethylene oxide), poly(propylene oxide), Polyquaterium-11, Polyquaterium-39, poloxamer, carbomer, gelatin, starch, alginic acid, salt of alginic acid, natural gums such as gum karaya, xanthan gum, guar gum, arabic gum tragacanth, or any combinations thereof.

The combination of the first and second polymers is preferably one or more of the following: a poly(vinylpyrrolidone)-alkyl vinyl ether/maleic anhydride copolymer, poly(vinylpyrrolidone)-alkyl vinyl ether/maleic acid copolymer, poly(vinylpyrrolidone)-alkali metal or an amine salt of alkyl vinyl ether/maleic acid copolymer, poly(vinylpyrrolidone)-partially or fully crosslinked alkyl vinyl ether/maleic anhydride copolymer, poly(vinylpyrrolidone)-vinyl acetate copolymer, poly(vinylpyrrolidone)-polyurethane interpolymer, poly(vinylpyrrolidone)-chitosan, poly(vinylpyrrolidone)-polyacrylates, poly(vinylpyrrolidone)-poly(acrylic acid), poly(vinylpyrrolidone)-poly(vinyl alcohol), poly(vinylpyrrolidone)-poly(vinyl alcohol-g-ethylene glycol) copolymer, poly(vinylpyrrolidone)-cellulose derivatives, poly(vinylpyrrolidone)-hydroxy-propyl-methyl cellulose, poly(vinylpyrrolidone)-hydroxy-ethyl cellulose, poly(vinylpyrrolidone)-hydroxypropyl cellulose, poly(vinylpyrrolidone)-poly(ethylene oxide), poly(vinylpyrrolidone)-poly(propylene oxide), poly(vinylpyrrolidone)-Polyquaterium-11, poly(vinylpyrrolidone)-Polyquaterium-39, poly(vinylpyrrolidone)-poloxamer, poly(vinylpyrrolidone)-carbomer, poly(vinylpyrrolidone)-gelatin, poly(vinylpyrrolidone)-starch, poly(vinylpyrrolidone)-alginic acid, poly(vinylpyrrolidone)-salt of alginic acid, poly(vinylpyrrolidone)-gum karaya, poly(vinylpyrrolidone)-xantham gum, poly(vinylpyrrolidone)-guar gum, poly(vinylpyrrolidone)-arabic gum, poly(vinylpyrrolidone)-tragacanth, or any combinations thereof.

Preferred commercial examples of such a first polymer includes poly alkyl vinyl ether-maleic acid copolymer (PVM/MA copolymer), such as, Gantrez AN 119, AN 139, S-97 and MS-955, poly(vinyl alcohol); poly(acrylic acid); Poloxamer 407 (Pluronic); poly(vinyl-pyrrolidone-co-vinylacetate) copolymer (PVPNA copolymer), such as Luviskol VA and Plasdone S630 PVPNA; poly(vinylpyrrolidone) (PVP, K-15 to about K-120); Polyquaterium-11 (Gafquat 755N); Polyquaterium-39 (Merquat plus 3330); carbomer (Carbopol); hydroxypropyl-methyl cellulose; hydroxyl-ethyl cellulose; hydroxypropyl-cellulose; gelatin; and alginate salt such as sodium alginate. Examples of the preferred polyurethanes include polycarbamyl polyglycol esters and poly(vinylpyrrolidone-polyurethane) interpolymer, such as, a poly(vinylpyrrolidone/polycarbamyl) polyglycol ester interpolymer.

The combination of the first and second polymers is a combination of PVP and a polymer. The polymers can favorably interact or complex to effectively increase the molecular weight of the polymer mixture. A linking agent can also be incorporated to promote the interaction between polymers. The effective increase in molecular weight results in a decrease in the rate of dissolution of the strip. Without being bound by any theory or structure, it is believed that the interaction between PVP and the polymers described in the examples, including polymers, such as, Gantrez, Aquamere, Chitosan and Pecogel, is accomplished through hydrogen bonding. Such interactions enable the polymer complex to bind onto the teeth and thereby provide effective whitening of the teeth.

Preferably, the water-soluble or water dispersible polymer system is present up to about 99.9 wt % based on the total weight of the whitening strip. More preferably, the polymer system is about 60 wt % to about 98 wt % of the total weight of the whitening strip.

Also, the amount of the first polymer is about 1 wt % to about 99 wt %, and preferably about 25 wt % to about 75 wt %, of the total weight of the whitening strip. The amount of the second polymer is about 1 wt % to about 80 wt %, and preferably about 5 wt % to about 50 wt %, of the total weight of the whitening strip.

The whitening agent, which is an active agent, in the strip can, upon contact with saliva, release the active agent onto the teeth in the oral cavity. Alternatively, the active agent can permeate through the film and be released to the surface where it is applied, including surfaces, such as, enamel, gum tissue and tongue.

The whitening agents that can be used in the present invention include hydrogen peroxide; carbamide peroxide; peroxycarbamate; persulfate, such as, persulfate salt or percarbonate salt; a perboric acid; perborate salt; PVP-hydrogen peroxide complex; calcium peroxide; metal chlorite (e.g. calcium chlorite, barium chlorite, magnesium chlorite, lithium chlorite, sodium chlorite, and potassium chlorite), hydroperoxide; peroxyacids; organic peroxides (e.g. benzoyl peroxide) chlorine dioxide; hydrogen peroxide adduct of carbodiimide persulfate; peroxide-generating compounds (e.g. azobisisobutyronitrile), phosphates, polyphosphates, alkali metal phosphates, alkali metal polyphosphates, sodium tripolyphosphate, or any combinations thereof.

The whitening agent is present up to about 99 wt % based on the total weight of the whitening strip. Preferably, the whitening agent is about 0.5 wt % to about 99 wt % of the total weight of the whitening strip. More preferably, the whitening agent is about 2 wt % to about 75 wt % of the total weight of the whitening strip.

In one preferred embodiment, the whitening agent is an alkali metal polyphosphate, such as sodium tripolyphosphate, and it is present in an amount about 7.5 wt % to about 25 wt % of the total weight of the whitening strip.

The whitening strip can also have an ingredient that further enhances benefits to the oral cavity and teeth. Such ingredients include: an antimicrobial agent, a mineralization compound, a stain prevention compound, a desensitization compound, an anti-calculus agent, a flavoring agent, an anti-inflammatory agent, an antioxidant, a volatile sulfur scavenger, an odorant neutralizer, and/or a vitamin. The whitening strip may also have a penetration enhancer, a plasticizer, a preservative, a surfactant or wetting agent, an anesthetic, an anti-allergenic, a pharmaceutical, or any combinations thereof. However, the whitening strip is preferably free of surfactants that are undesired in the oral cavity.

The following antimicrobial agents can preferably be used in the present whitening strip: polyphenols (e.g. triclosan) zinc salts, stannous fluoride, chlorhexidine, hexetidine, sanguinarine, benzalkonium chloride, salicylanilide, domiphen bromide, cetylpyridinium chloride, tetradecylpyridinium chloride (TPC), N-tetradecyl-4-ethylpyridinium chloride (TDEPC), octenidine, delmopinol, octapinol, and other piperidine derivatives, nicin preparations, zinc/stannous ion agents, antibiotics such as augmentin, amoxicillin, tetracycline, doxycycline, minocycline, and metronidazole, and analogs and salts of the above, essential oils including thymol, menthol, eugenol, geraniol, carvacrol, citral, hinokitiol, eucalyptol, catechol, methyl salicylate, hydrogen peroxide, metal salts of chlorite, or any combinations of all of the above.

The following mineralization compounds are preferred for use in the present whitening strip: sodium monofluorophosphate, potassium monofluorophosphate, magnesium monofluorophosphate, acidulated fluorophosphate, amine fluoride, water-soluble salts of fluoride, such as, sodium fluoride, potassium fluoride, calcium fluoride, stannous fluoride, sodium fluorosilicate, bis-salicylato-bis-fluorotitanium (IV), ammonium fluorosilicate, calcium salt, phosphate salt, calcium salt/phosphate salt, calcium salt/ionic fluoride sources, zinc salt/phosphate salt), or any combinations thereof.

The following desensitization compounds can preferably be used in the present whitening strip: water-soluble potassium salt including potassium nitrate, potassium citrate, potassium chloride, potassium bicarbonate, potassium oxalate, and tubular occlusion compounds (e.g., ferric oxalate), or any combinations thereof.

The following anti-calculus agents can preferably be used in the present whitening strips: phosphates, pyrophosphates, polyphosphates, phosphonates (e.g. ethane-1-hydroxy-1,1-diphosphonate, 1-azacycloheptane-1,1-diphosphonate) polyphosphonates, polyacrylates and other polycarboxylates, ethylenediaminetetraacetic acid and other calcium chelators, carboxylic acids and their salts, zinc salts (e.g. sodium zinc citrate), PVM/MA copolymer or other polymers which interfere with crystal nucleation or growth, or any combinations thereof. It should be understood that when phosphate anti-calculus agent is used in conjunction with phosphate whitening agent, the phosphate anti-calculus agent(s) used is typically different than the phosphate whitening agent(s) used.

The following flavoring agents can preferably be used in the present whitening strip: flavoring oils, e.g., oils of spearmint, peppermint, wintergreen, sassafras, clove, sage, eucalyptus, marjoram, cinnamon, lemon, menthol, anethole, thymol, parsley oil, oxanone and orange, alpha-irisone, cassia, marjoram, oils thereof, propenyl guaethol, and methyl salicylate. Sweetening agents including, but not limited to, sucrose, lactose, maltose, sorbitol, xylitol, sodium cyclamate, sucralose, acesulfame-K, aspartame, and sodium saccharin. Any combinations of the preceding flavoring agents are also suitable for use in the whitening strip.

The following anti-inflammatory agents can preferably be used in the present whitening strips: non-steroidal anti-inflammatory agents, such as, ketorolac, flurbiprofen, ibuprofen, naproxen, indomethacin, aspirin, ketoprofen, piroxicam, meclofenamic acid, or any combinations thereof. Also, steroidal and non-steroidal anti-inflammatory agents and plant extracts that have demonstrated anti-inflammatory activities can be used.

The following antioxidants can preferably be used in the present whitening strips: Vitamin E, ascorbic acid, Uric acid, kojic acid, coenzyme compounds (e.g. coenzyme Q-10), carotenoids, Vitamin A, flavonoids and polyphenols, herbal antioxidants, melatonin, aminoindoles, lipoic acids, or any combinations thereof.

Other suitable antioxidants include: rosemary extract, tocopherol, a derivative of tocopherol including a tocotriene, carotene, a carotenoid, a phenolic antioxidant including a phenolic acid, a bioflavonoid, a plant extract, curcumin, tetrahydrocurcumin, camphorol, quercetine, epigenine, or any mixtures thereof.

The following vitamins can preferably be used in the present whitening strips: Vitamin K, retinol (vitamin A), tocopherol, or any combinations thereof.

The whitening strip can be prepared by solution deposition, film-casting, dye-casting or extrusion.

In operation, upon contact with the teeth and moisture, the strip, due to the polymer system, hydrates in such a manner as to conform to the contours of the teeth and the interstices, filling any space it encounters. As a result, the interaction of the whitening composition with the surface of the teeth and the interstices between the teeth becomes more intimate and stronger over time. Applicants have surprisingly found that, following initial application of the strip, the adhesive properties of the strip continue to improve thereby providing better, intimate contact of the strip with and adhesion to the morphology of the teeth.

It should be noted that unlike the strips described in the prior art, the whitening strip of the present invention does not require a backing layer. In fact, the whitening strip, according to the present invention, preferably does not have a backing layer.

Preferably, after bringing the whitening strip into contact with the teeth, the whitening action of the strip is completed within less than about 60 minutes. Typically however, contacting the teeth with the whitening strip for a period of time from about 1 minute to about 25 minutes is sufficient to whiten the teeth when used as directed in a whitening treatment regimen.

The present invention further provides a process for preparing a whitening strip in the form of a dry film. The process has the steps of combining a whitening agent, a water-soluble or water dispersible polymer system that provides a stabilized matrix for the whitening agent, and a volatile solvent, to form a mixture; applying the mixture onto a surface to form a film of the mixture on the surface; and removing the volatile solvent to produce the whitening strip in the form of a dry film.

The volatile solvent can be any commonly used volatile solvent. However, as noted in the following Examples, the preferred volatile solvent is either water or ethanol.

As noted below in the Examples, wetting agents, such as Pluronic F-68 can be used, and may remain in the final whitening strip, to improve spreading and film forming ability.

The present invention also provides a method of whitening teeth. The method includes the step of applying onto the teeth a whitening strip for a period of time sufficient to produce a detectable improvement in the whiteness of the teeth. The method of the present invention does not require the removal of the strip since the strip dissolves in the mouth.

The Examples that follow are illustrative of the present invention. They should not be construed as being limiting in any manner.

### EXAMPLE 1

| | Formulation | Dry Film |
|---|---|---|
| Component | % w/w | % w/w |
| Aquamere A-1212 | 39.4 | 74.68 |
| Ethanol | 39.5 | — |
| Plasdone K-90 | 10 | 18.95 |
| Hydrogen Peroxide (30%) | 11.2 | 6.37 (as H2O2) |

Aquamere is a polyurethane ester.

### EXAMPLE 2

| | Formulation | Dry Film |
|---|---|---|
| Component | % w/w | % w/w |
| Water | 64.8 | — |
| Carbamide peroxide | 10 | 28.41 |
| Gantrez MS-955 | 9 | 25.57 |
| Glycerin | 8 | 22.73 |
| Plasdone K-90 (PVP) | 8 | 22.73 |
| Pluronic F-68 | 0.1 | 0.28 |
| Citric acid | 0.05 | 0.14 |
| EDTA | 0.05 | 0.14 |

Pluronic F-68 was added to reduce surface tension and improve spreading and film forming. It can be eliminated depending on the substrate on which the film is cast.

Plasdone K-90 was dissolved in a solution of water, carbamide peroxide, glycerin, pluronic F-68, EDTA, and citric acid. Films were dried at room temperature overnight followed by drying at +37° C. ca. 30 minutes.

### EXAMPLE 3

| | Formulation | Dry Film |
|---|---|---|
| Component | % w/w | % w/w |
| Water | 85.4 | — |
| Plasdone K-90 | 9.6 | 81.01 |
| Chitosan | 1 | 8.44 |
| H2O2 (30% w/w) | 4 | 10.13 |
| | | (as H2O2) |
| Disodium pyrophosphate | 0.05 | 0.42 |

Plasdone K-90 was added as a 20% w/w solution in 0.1 M NaOH, Chitosan was added as a 2% w/w solution in 0.1 M NaOH Films were dried at room temperature overnight.

### EXAMPLE 4

### Peroxydone K-90/Gantrez MS-955/PEG400

| | Formulation | Dry Film |
|---|---|---|
| Component | % w/w | % w/w |
| Water | 75.8 | — |
| Peroxydone K-90 | 10 | 42.7 |
| Gantrez MS-955 | 9 | 38.4 |
| PEG 400 | 4 | 17.1 |
| Hydrogen peroxide (30%) | 1.1 | 1.4 |
| Citric acid | 0.05 | 0.2 |
| EDTA | 0.05 | 0.2 |

### EXAMPLE 5

### Peroxydone K-90/Pecogel (PVP/Polycarbamyl Polyglycol Ester)

| | Formulation | Dry Film |
|---|---|---|
| Component | % w/w | % w/w |
| Ethanol | 46.2 | — |
| Pecogel A-12 (12% w/w sol) | 41.7 | 29.2 |
| Peroxydone K-90 | 10 | 58.5 |
| Plasdone K-90 | 2 | 11.7 |
| Citric acid | 0.05 | 0.3 |
| EDTA | 0.05 | 0.3 |

### EXAMPLE 6

| | Formulation | Dry Film |
|---|---|---|
| Component | % w/w | % w/w |
| Ethanol | 62.9 | — |
| Pecogel A-115(20% w/w sol) | 25 | 29.2 |
| Peroxydone K-90 | 10 | 58.5 |
| Plasdone K-90 | 2 | 11.7 |
| Citric acid | 0.05 | 0.3 |
| EDTA | 0.05 | 0.3 |

### EXAMPLE 7

### Peroxydone K-90/Chitosan

| | Formulation | Dry Film |
|---|---|---|
| Component | % w/w | % w/w |
| Water | 85.1 | — |
| Peroxydone K-90 | 8.8 | 59.1 |
| Plasdone K-90 | 2.5 | 16.8 |
| Chitosan (Medium MW) | 1 | 6.7 |
| Glycerin | 2.5 | 16.8 |
| Citric acid | 0.05 | 0.3 |
| EDTA | 0.05 | 0.3 |

*Based on the theoretical value assuming there are no losses during preparation.

### EXAMPLE 8

| | Formulation | Dry Film |
|---|---|---|
| Component | % w/w | % w/w |
| Ethanol | 78.84 | — |
| Sodium | 1.57 | 7.42 |
| tripolyphosphate (STP) | | |
| Glycerin | 0.51 | 2.41 |
| Klucel G | 3.0 | 14.16 |
| Plasdone K-29/32 | 16.09 | 76.01 |

In another embodiment of the present invention polymeric dissolvable films containing different levels of sodium tripolyphosphate (STP) were made and tested for their in vitro whitening performance. Example 8 above exemplifies such a film. The films were cast from ethanolic solutions of poly(vinyl pyrrolidone) (PVP), hydroxypropylcellulose (HPC) and glycerin. Phosphate content was determined by ion chromatography. Whitening performance was assessed using an in vitro model employing tea stained bovine teeth. Films were applied to teeth (n=7) for 30 min in the presence of PBS substantivity challenge. Tooth color was measured at baseline and following 2, 4 and 6 treatments using a Labscan chromometer. Change in color was presented as ΔL*.

Films formulated containing different levels of STP were highly bioadhesive and exhibited good mechanical properties. The mean± standard deviation values for ΔL* after 6 treatments were: 1.84±1.24, 1.67±0.95, 1.95±0.57 and 2.85±1.23 for films containing 0, 2.4, 4.9 and 7.4% w/w STP, respectively. There was no statistically significant difference between the whitening performance of films with 0, 2.4 or 4.9% w/w STP. However, it was surprisingly found that ΔL* values for films with about 7.4% w/w STP were statistically significantly higher than the rest after six treatments. Phosphate content remained unchanged after four weeks storage at 40° C./75% RH; however, a small fraction of STP was transformed into pyrophosphate, as expected.

Surprisingly, significant in vitro whitening was obtained in the absence of peroxide by using substantive dissolvable strips containing STP. Low concentrations of STP (i.e. 2.4 and 4.9% w/w) did not show any whitening advantage compared to that of the placebo. However, about 7.5% w/w STP films exhibited significant in vitro whitening. Therefore, it is believed that STP in an amount between about 7.5% w/w to about 25% w/w, based on the weight of the whitening strip, will achieve the desired and unexpected tooth whitening effect.

The present invention also provides a multi-layered stack, i.e., a multi-layered whitening strip for whitening teeth. The stack has a first controlled-dissolution, water-soluble or water dispersible whitening layer or film having a first whitening agent and a first water-soluble polymer system; and a second controlled-dissolution water-soluble or water dispersible whitening layer or film disposed on the first film and having a second agent (e.g. an accelerant) and a second water-soluble or water dispersible polymer system. The first and the second films are joined to form a stack. Adhesives may or may not be necessary to join the two layers. The overall dissolution of the strip in the oral cavity is controlled by the composition of each individual layer.

Multi-layered stacks can be formed from cast or extruded films, or any combination of cast and extruded films. Other means of depositing a discreet layer or domain (e.g., depositing spray dried materials) may also be used. The layers can have the same or different compositions and can be organized in the stack in any manner or in any form, including random and/or ordered arrangements and repeating units.

Each individual layer within the strip can be made of identical ingredients or the ingredients in each layer can be varied to provide enhanced and/or multiple benefits.

An example of a benefit provided by the multi-layered strip is the ability to provide a stable delivery system for incompatible ingredients. Incompatible ingredients can be separated within discreet domains until the strip is hydrated during use.

The multi-layer whitening strip can also have one or more layers of the first and/or the second controlled-dissolution water-soluble or water dispersible whitening films in single or repeating units.

Further, each of the layers of the first and/or the second controlled-dissolution water-soluble or water dispersible whitening films can have different formulations to provide different rates of dissolution. The multilayer strip can be designed to provide a directional diffusion of the whitening agents from the multi-layer strip to the teeth by selection of layers with varying dissolution rates.

Still further, each layer can have one or more zones. A zone is a region in a layer. In an example of this embodiment, a strip can have a zone containing a desensitizing agent, preferably about the periphery of the strip.

Additionally, the multi-layered strip can have peroxide containing films and bleaching accelerators separated in the particular layers, which, upon dissolution of the strip, can accelerate whitening without the need for pre-mixing of whitening agents and accelerators. This layered system provides a stable environment for bleaches and accelerators within the same strip.

The multi-layered strips or films can also promote mineralization, which can be helpful in preventing tooth sensitivity. Mineralization in this manner can also help to increase the opacity of the teeth and subsequently render the teeth whiter in appearance.

The multi-layered strips that allow different rates of the active diffusion of the ingredient can promote unidirectional delivery of the active to the desired site. For example, if such devices are applied to the teeth to promote delivery directly to the tooth, less active ingredient would be lost into the oral cavity.

Among the benefits of multi-layered strips is the ability to combine in the layer or film, ingredients that are normally incompatible thereby providing such incompatible ingredients in a single delivery system.

In one embodiment, peroxide activation can be achieved with, for example, a dual layered film, one layer containing the peroxide, and the other layer containing an activator, such as, a pH adjuster.

In another embodiment, different ingredients can be placed in separate film domains until hydration in the oral cavity causes them to combine.

In still another embodiment, films or layers with differing dissolution rates can be developed to promote direction of an agent so that the agent is delivered to the surface of attachment, for example, peroxide into enamel, or out into the oral cavity, for example, slow release flavor delivery system.

Another potential advantage of multi-layered films is that layers can contain materials, such as, polymer systems that can be readily solubilized in the layer they reside. However, the solubility of these materials can be reduced with components in separate layers to promote a formation of desirable residues on surfaces. This can result in a lingering benefit, for example, long lasting flavor or a sensitivity treatment of prolonged duration.

While the utility of the films or layers according to the present invention is generally described with respect to oral care and treatment, such films or layers can be used in denture care products, smoking cessation products, dermatological treatments and systems for general drug delivery through buccal surfaces in the oral cavity.

The whitening strip, whether a single or multi-layer strip, can have two or more zones or domains on the surface of a layer of the strip. A zone is an area on a layer of a whitening strip that can separate ingredients (whether compatible or incompatible ingredients) from one another until the ingredients are combined during dissolution of the whitening strip. Thus, each zone can provide an area on the whitening strip that has a specific ingredient that can provide a specific benefit, such as, for example, anti-irritation or an anti-hypersensitivity ingredient along the gingival margin.

It should be understood that the foregoing description is only illustrative of the present invention. Various alternatives and modifications can be devised by those skilled in the art without departing from the present invention. Accordingly, the present invention is intended to embrace all such alternatives, modifications and variations that fall within the scope of the appended claims.

1. A dissolvable, strip for whitening teeth in an oral cavity, comprising:
a phosphate whitening agent; and
a water-soluble or water dispersible polymer system having a poly(vinylpyrrolidone) or any derivative thereof or any of its copolymers, and at least one other polymer, wherein said strip will dissolve by saliva in an oral environment.

2. The whitening strip of claim 1, wherein said phosphate whitening agent is selected from the group consisting of alkali metal phosphate, polyphosphate, alkali metal polyphosphate, and any combinations thereof.

3. The whitening strip of claim 1, wherein said phosphate whitening agent is sodium tripolyphosphate.

4. The whitening strip of claim 1, wherein said whitening agent is about 2 wt. % to about 75 wt. % of the total weight of the whitening strip.

5. The whitening strip of claim 3, wherein said sodium tripolyphosphate is about 7.5 wt. % to about 25 wt. % of the whitening strip.

6. The whitening strip of claim 1, wherein said whitening agent is encapsulated in a water-soluble or water dispersible shell.

7. The whitening strip of claim 6, wherein said water-soluble or water dispersible shell controls the dissolution of said whitening composition by interaction of the shell with saliva.

8. The whitening strip of claim 1, wherein said water-soluble or water dispersible polymer system is selected from the group consisting of a poly(vinylpyrrolidone)-alkyl vinyl ether/maleic anhydride copolymer, poly(vinylpyrrolidone)-alkyl vinyl ether/maleic acid copolymer, poly(vinylpyrrolidone)-alkali metal or an amine salt of alkyl vinyl ether/maleic acid copolymer, poly(vinylpyrrolidone)-partially or fully crosslinked alkyl vinyl ether/maleic anhydride copolymer, poly(vinylpyrrolidone)-vinyl acetate copolymer, poly(vinylpyrrolidone)-polyurethane interpolymer, poly(vinylpyrrolidone)-chitosan, poly(vinylpyrrolidone)-poly(acrylic acid), poly(vinylpyrrolidone)-poly(vinyl alcohol), poly(vinylpyrrolidone)-poly(vinyl alcohol-g-ethylene glycol) copolymer, poly(vinylpyrrolidone)-cellulose derivatives, poly(vinylpyrrolidone)-hydroxy-propyl-methyl cellulose, poly(vinylpyrrolidone)-hydroxy-ethyl cellulose, poly(vinylpyrrolidone)-hydroxy-propyl cellulose, poly(vinylpyrrolidone)-poly(ethylene oxide), poly(vinylpyrrolidone)-poly(propylene oxide), poly(vinylpyrrolidone)-Polyquaterium-11, poly(vinylpyrrolidone)-Polyquaterium-39, poly(vinylpyrrolidone)-poloxamer, poly(vinylpyrrolidone)-carbomer, poly(vinylpyrrolidone)-gelatin, poly(vinylpyrrolidone)-starch, poly(vinylpyrrolidone)-alginic acid, poly(vinylpyrrolidone)-salt of alginic acid, poly(vinylpyrrolidone)-gum karaya, poly(vinylpyrrolidone)-xantham gum, poly(vinylpyrrolidone)-guar gum, poly(vinylpyrrolidone)-arabic gum, poly(vinylpyrrolidone)-tragacanth, poly(vinylpyrrolidone)-polyvinylpyrrolidone derivatives or copolymers, and any combinations thereof.

9. The whitening strip of claim 8, wherein said poly(vinylpyrrolidone)-polyurethane interpolymer is a poly(vinylpyrrolidone)/polycarbamyl polyglycol ester interpolymer.

10. The whitening strip of claim 1, wherein said at least one other water-soluble or water dispersible polymer is selected from the group consisting of an alkyl vinyl ether/maleic anhydride copolymer, alkyl vinyl ether/maleic acid copolymer, alkali metal or an amine salt of alkyl vinyl ether/maleic acid copolymer, partially or fully crosslinked alkyl vinyl ether/maleic anhydride copolymer, vinyl acetate copolymer, polyacrylates, polyurethane interpolymers, chitosan, poly(acrylic acid), poly(vinyl alcohol), poly(vinyl alcohol-g-ethylene glycol) copolymer, cellulose derivatives, hydroxy propyl-methyl cellulose, hydroxyl-ethyl cellulose, hydroxy-propyl cellulose, poly(ethylene oxide) or copolymers thereof, poly(propylene oxide) or copolymers thereof, Polyquaterium-11, Polyquaterium-39, poloxamer, carbomer, gelatin, starch, alginic acid, salt of alginic acid, gum karaya, xanthan gum, guar gum, arabic gum tragacanth, and any combinations thereof.

11. The whitening strip of claim 1, wherein said water-soluble or water dispersible polymer system is present up to about 99.9 wt % based on the total weight of the whitening strip.

12. The whitening strip of claim 1, wherein said water-soluble or water dispersible polymer system is about 60 wt % to about 95 wt % of the total weight of the whitening strip.

13. The whitening strip of claim 1, wherein said water-soluble or water dispersible polymer system is also hydratable on contact with saliva.

14. The whitening strip of claim 13, wherein the whitening strip adapts to the morphology of the teeth upon contact with saliva in the oral cavity.

15. The whitening strip of claim 1, wherein the whitening strip is free of surfactants.

16. The whitening strip of claim 1, further comprising:
an ingredient selected from the group consisting of an antimicrobial agent, a mineralization compound, a desensitization compound, an anti-calculus agent, a flavoring agent, an anti-inflammatory agent, an antioxidant, a stain prevention agent, and any combinations thereof.

17-20. (canceled)

21. The whitening strip of claim 1, further comprising a plasticizer.

22-25. (canceled)

26. The whitening strip of claim 1, wherein the whitening strip is free of a backing.

27. The whitening strip of claim 1, wherein said strip is prepared by a method selected from the group consisting of solution deposition, film-casting, dye-casting and extrusion.

28. The whitening strip of claim 1, wherein the whitening strip is a single layer.

29. The whitening strip of claim 28, wherein the layer has a surface with two or more zones.

30. The whitening strip of claim 29, wherein each one of the two or more zones have different compositions.

31-36. (canceled)

37. A dissolvable, multi-layer strip for whitening teeth in an oral environment, comprising:
a first controlled-dissolution film having a first whitening agent and a first water-soluble or water dispersible polymer system; and
a second controlled-dissolution film disposed on said first film and having a second whitening agent and a second water-soluble or water dispersible polymer system,
wherein said first and said second films are joined to form a stack, and
wherein the dissolution of each of said first and said second films is controlled by interaction of said first and second films with saliva in the oral environment.

38-52. (canceled)

53. A process for preparing a whitening strip in the form of a dry film, comprising:
combining a phosphate whitening agent, a water-soluble or water dispersible polymer system for said whitening agent and a volatile solvent, to form a mixture;
applying said mixture onto a surface to form a film of said mixture on said surface; and
removing said volatile solvent to produce a whitening strip in the form of the dry film.

54. A method of whitening teeth, comprising:
applying onto the teeth whitening strips as a regimen specified in the direction for use according to claim 1 for a period of time sufficient to produce a detectable improvement in the whiteness of the teeth.

55-61. (canceled)
